(19) United States
(12) Patent Application Publication
(10) Pub. No.: US 2023/0266060 A1
(43) Pub. Date: Aug. 24, 2023

(54) METHOD AND SYSTEM FOR SEPARATING A FEED FLOW

(71) Applicant: Linde GmbH, Pullach (DE)

(72) Inventors: Bernd KLEIN, München (DE); Torben HÖFEL, München (DE)

(21) Appl. No.: 18/004,260

(22) PCT Filed: Jun. 22, 2021
(86) PCT No.: PCT/EP2021/025223

§ 371 Date: Jan. 4, 2023

(30) Foreign Application Priority Data
Jul. 7, 2020 (DE) 10 2020 117 937.5

(51) Int. Cl. F25J 3/06

(52) U.S. Cl. CPC F25J 3/062; F25J 3/0645; F25J 3/065; F25J 3/0655; F25J 2210/04; F25J 2210/62; F25J 2215/02; F25J 2215/64; F25J 2215/66; F25J 2230/30; F25J 2240/02

(57) ABSTRACT

A method and a system for separating a feed flow which contains at least hydrogen and a hydrocarbon with three or four carbon atoms per molecule, in particular propane, propylene, propadiene, butane, 1-butene, 2-butene, and/or 1,3-butadiene. The condensed feed flow is cooled over multiple cooling steps in at least two heat exchangers and is then separated into a condensate and a residual gas flow after each cooling step. The at least two heat exchangers are operated at least two different temperature levels, wherein a hot heat exchanger is operated at an average temperature level, and a cold heat exchanger is operated at a lower temperature level. An internal refrigerant which is made of a part of one of the condensate flows and a part of one of the residual gas flows, is used to dispense a part of the heat from the cold heat exchanger.

The invention relates to a method and to a system for separating a feed flow according to the preambles of the independent claims.

### PRIOR ART

In the industrial production and processing of hydrocarbons and other organochemical compounds, it is frequently necessary to separate product flows of a method step into different components before further steps can be carried out, for example in order to separate such components which interfere in the following steps or to emit a product with a required purity.

For such separations, so-called cryogenic separation methods are frequently used, in which a gaseous feed flow is cooled, wherein the feed flow is at least partially liquefied. By means of such partial condensations, various components present in the feed flow can be separated from one another in accordance with their respective boiling points or vapor pressures at the respectively prevailing pressures or temperatures. For this purpose, so-called C2-refrigerants are frequently used which consist of hydrocarbon mixtures which are composed essentially of compounds having two carbon atoms per molecule. The use of pure C2-refrigerants such as ethane or ethylene is also possible.

Furthermore, separation methods are known which operate without C2 refrigerants. Such a cryogenic separation method is known, for example, from U.S. Pat. No. 6,333,445 B1. There, a gaseous feed flow originating from an alkane dehydrogenation process and which therefore contains hydrogen and the unconverted alkane and an alkene produced therefrom is first compressed and then cooled and partially condensed using two heat exchangers. A liquid raw material flow containing the alkane required for the dehydrogenation process is evaporated and heated as the coolant. A “feed flow” is understood below to mean a gaseous flow which is fed to a separation method. This originates from an organochemical conversion method, to which in turn a “raw material flow” is fed, according to the terminology used below. In the example explained, the raw material flow is thus fed to the alkane dehydrogenation process and converted therein into the feed flow of the separation process.

The condensates or liquid flows produced from the feed flow are separated from the remaining residual gas flows. The cooling capacity is applied primarily by the evaporation heat absorbed by the liquid raw material flow. The process conditions, in particular a positive pressure difference between the gaseous feed flow and the liquid raw material flow, bring about a temperature difference and thus enable the heat transfer and the partial condensation of the feed flow or evaporation of the raw material flow.

Depending on the configuration of the alkane dehydrogenation process, a situation can thereby arise in which the amount of heat which is withdrawn from the evaporation of the raw material flow is not sufficient to achieve the separation or partial condensation of the feed flow.

The object of the present invention is therefore to provide an improved separation method which, without C2 refrigerant, ensures a corresponding separation even in unfavorable situations with respect to the amount of heat withdrawn by the evaporation of the raw material flow.

### DISCLOSURE OF THE INVENTION

This object is achieved by methods and systems according to the respective independent claims. Advantageous designs and developments result from the features of the dependent claims and from the following description.

Before the features and advantages of the invention are shown, their principles and terms used within the scope of the disclosure will be explained.

A “compressor” is a device which is configured for compressing at least one gaseous flow from at least one inlet pressure (also referred to as suction pressure) at which it is supplied to the compressor to at least one end pressure at which it is withdrawn from the compressor. A compressor forms a structural unit which, however, can have several “compressor stages” in the form of rows of pistons, screws and/or blades (i.e., axial or radial compressor stages). In particular, corresponding compressor stages are driven by means of a common drive, for example via a common shaft. A compressor in the described sense or a compressor stage of such a compressor performs a “compression step” according to the terminology of the present disclosure.

A “cooler” is a device which is configured for cooling a (for example gaseous or liquid) fluid flow from at least one inlet temperature at which it is supplied to the cooler to at least one end temperature at which it is withdrawn from the cooler. A cooler forms a structural unit which, however, can have several “cooling stages” in the form of (e. g., plate, pipe, counter-flow) heat exchangers and/or expanders (for example throttle valves or turbines). In particular, corresponding cooling stages can be realized using a single heat exchanger. A cooler in the described sense or a cooling stage of such a cooler performs a “cooling step” in the terminology of this disclosure.

A “thermal separation” is characterized in the language of this disclosure in that a gas mixture is separated under at least partial liquefaction in the same, and in this case a suitable refrigerant is used. Known heat exchangers are used for this purpose. The separation is effected by means of known phase separation devices, for example by means of gas separators. In a thermal separation, in particular so-called C3 and/or C2-refrigerants are used. These are conducted between different pressure levels, wherein the aforementioned compressors and, for example, known expansion turbines or expansion or throttle valves are used.

If in the present disclosure it is stated that a flow or a mixture of substances is “enriched” with one or more components in relation to another flow or another mixture, this is to be understood in such a way that the concentration of this/these component(s) in the flow or mixture enriched in this way is higher by at least a factor of 1.1, 1.2, 1.5, 2, 3, 5, 10, 30, 100, 300 or 1000 in comparison to the reference flow or mixture. A “depleted” material flow accordingly has a lower concentration than the reference flow and in particular has a concentration of the component which is lower compared to the reference flow, i.e., at most 90%, 80%, 50%, 30%, 10%, 3%, 1%, 0.3% or 0.1% of the concentration of the component in the reference flow.

The present disclosure uses the terms “pressure level” and “temperature level” to characterize pressures and temperatures, which means that corresponding pressures and temperatures in a corresponding plant do not have to be used in the form of exact pressure or temperature values in order to realize the inventive concept. However, such pressures and temperatures typically fall within certain ranges that are, for example, 1%, 5%, 10%, 20% or even 50% around an average. In this case, corresponding pressure levels and temperature levels can be in disjointed ranges or in ranges that overlap one another. In particular, pressure levels, for example, include unavoidable or expected pressure losses. The same applies to temperature levels.

If it is stated in this description that a mixture contains at least one liquid phase, this is to be understood as meaning that the mixture can contain one or more liquid phases which are completely, partly or not miscible with one another.

### ADVANTAGES OF THE INVENTION

In the following, features and advantages of the invention are explained primarily with respect to the method mentioned. The corresponding statements also apply analogously to systems according to the invention and advantageous embodiments thereof, which profit accordingly from the advantages. For example, it can be mentioned that a flow is subjected to a method step. This is to be understood in relation to a corresponding system in such a way that components which are designed to carry out a corresponding method step are provided and means, for example pipelines, valves and the like, are provided for feeding the respective flow into the component. The explanations relating to a system accordingly also apply to a corresponding method.

According to the invention, a method for separating a feed flow comprising at least hydrogen and a hydrocarbon having three carbon atoms per molecule is proposed. In the compressed state, the feed flow is partially liquefied by means of at least two coolers, which are operated at different temperature levels, via at least two cooling steps to obtain at least one first and one second condensate flow and at least one first and one second residual gas flow. The residual gas flow of a cooling step is respectively fed into the subsequent cooling step. In the case of partial liquefaction, each condensate flow is depleted with regard to hydrogen and enriched with respect to hydrocarbon relative to the feed flow and each residual gas flow is enriched with respect to hydrogen and depleted with respect to the hydrocarbon in relation to the feed flow. A liquid C3 product flow predominantly consisting of the hydrocarbon is formed from the condensate flows and a gaseous gas product flow consisting predominantly of hydrogen is formed using at least one of the residual gas flows. A part of at least one of the condensate flows is combined with a part of at least one of the residual gas flows and used as an internal refrigerant for at least one of the cooling steps (or coolers) under expansion. The expansion can take place before and/or after the combination.

Analogously to the method described above, in the method according to the invention, a raw material flow is also supercooled in at least one of the coolers, combined with a part of the gas product flow, expanded and used as refrigerant for the cooler. The advantage of the invention over the conventional method is that, irrespective of the amount, the pressure and the composition of the raw material flow, the separation of the feed flow can always be carried out, since the energy balance can be closed by means of compression of the feed flow upstream of the cooler and by cooling to a natural ambient temperature level. The expanded internal refrigerant can advantageously be returned to the feed flow, optionally with further gaseous extraction flows, before it is compressed. As a result, the method can be controlled significantly more flexibly than conventional methods and can, for example, be adapted to fluctuating amounts in terms of gaseous feed flow and/or liquid raw material flow and other fluctuating process conditions such as, for example, unfavorable pressure and/or temperature levels, which impede the heat exchange between condensing feed flow and evaporating raw material flow.

Preferably, at least one first cooling step takes place from a high inlet temperature level (for example temperature of the natural atmosphere or environment, e.g., 10° C. to 50° C.) to an average temperature level which is in a range from −40° C. to 10° C., preferably from −40° C. to −10° C., and at least one second cooling step to a low temperature level, which is in a range from −130° C. to 80° C., preferably from −110° C. to −90° C. This makes it possible to substantially separate the hydrocarbon from the hydrogen already at the average temperature level, for example in the first condensate flow.

In some embodiments of the invention, only flows of materials formed from the liquid raw material flow are used to achieve the low temperature level. In order to achieve the average temperature level, an externally generated or externally supplied refrigerant can additionally be used. As a result, any available process cooling can advantageously be integrated and the energy balance can be closed in a simple manner without requiring refrigerant at the low temperature level.

Advantageously, the first and second cooling steps take place by counterflow heat exchange, in particular of the condensing feed flow and the evaporating raw material flow. As a result, the energy balance can be closed essentially within the method.

Preferably, the second residual gas flow of the second cooling step is subjected to at least one expansion to obtain further condensate and residual gas flows. As a result, energy can be extracted from the method and this can be used in the form of mechanical energy, for example in order to drive pumps or compressors.

The internal refrigerant is preferably formed from a part of the second condensate flow and a part of the residual gas flow which has been expanded. As a result, the obtained internal refrigerant has particularly advantageous process conditions with respect to temperature and composition and can be optimally used for the supporting cooling of the second condensate flow.

Advantageously, several or all condensate flows are combined to form an aggregate flow, and the aggregate flow is subjected to a gas separation forming a flash gas and the C3 product flow. This results in synergy effects and potential savings in relation to required pipelines, insulations and the like.

In particular, the C3 product flow, the gas product flow and the flash gas are heated to a temperature level corresponding to the temperature level of the feed flow. As a result, no cold is lost, which is particularly favorable in terms of energy.

Since it is desired both for the flash gas and for the internal refrigerant to return the fluid back into the feed flow in order to increase the yield of the method, these two flows can be combined at a suitable point and fed back together to the feed flow before it is compressed.

The gas product flow is preferably used at least in part for the provision of the feed flow, in particular by mixing the gas product flow at least partially into the raw material flow. This is particularly advantageous when hydrogen is required as diluent medium for the production of the feed flow from the raw material flow in the course of the dehydrogenation process. This hydrogen does not have to be supplied from a separate source, but can be provided from the gas product which is produced in any case. By mixing a part of the gas product flow into the raw material flow at the lowest temperature level (after supercooling of the raw material flow), the raw material flow can otherwise be used more efficiently for cooling the feed flow.

A system according to the invention for separating a feed flow comprising at least hydrogen and a hydrocarbon having three carbon atoms per molecule has at least two heat exchangers, at least one of which can be operated at an average temperature level and at least one at a low temperature level, and which are configured to cool the feed flow according to the counterflow principle. In addition, it comprises at least two phase separation devices which are each configured to split a partially liquefied flow into a condensate flow and a residual gas flow, as well as means which are designed to combine a part of at least one of the condensate flows with a part of at least one of the residual gas flows to form an internal refrigerant and to supply the internal refrigerant after expansion to at least one of the heat exchangers. The expansion takes place before or after the combination or merging of the respective condensate and residual gas flow. The system is thus essentially configured to carry out a method according to the invention.

Preferred embodiments and further aspects of the invention and their advantages are explained in more detail below with reference to the attached drawing.

### BRIEF DESCRIPTION OF THE FIGURES

FIG. 1 schematically shows a preferred embodiment of a system according to the invention.

### EMBODIMENTS OF THE INVENTION

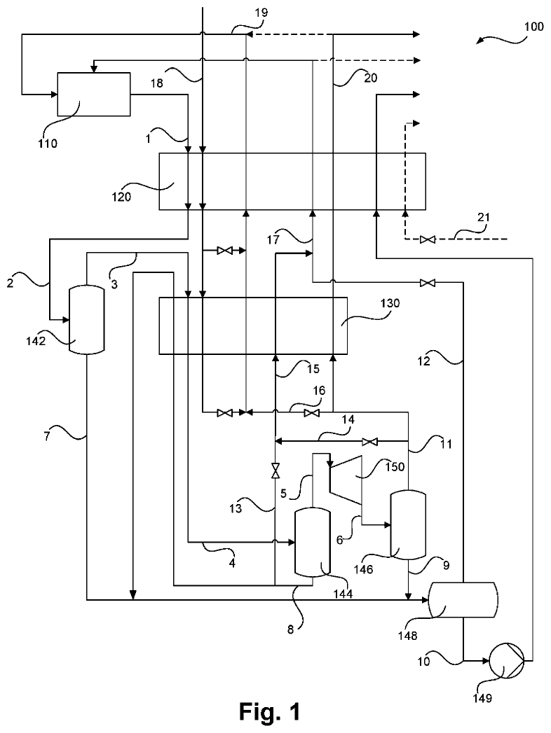

FIG. 1 schematically illustrates a system 100, which is configured to carry out a method according to the invention. The system 100 comprises, among other things, a hot heat exchanger 120, a cold heat exchanger 130 and a plurality of gas separators 142, 144, 146, 148.

Furthermore, in the example shown, the system 100 comprises a provision unit 110 which is designed to generate a feed flow 1 which contains at least hydrogen and a hydrocarbon with three or four carbon atoms per molecule (here and in the following, the latter is also referred to as C3 in relation to three carbon atoms per molecule, for example). In particular, the carbon is propene. The provision unit 110 can, for example, be designed as a reactor which is configured to carry out a propane dehydrogenation reaction. For this purpose, the reactor 110 is equipped with a catalyst and is charged with one or more raw material flows 18, 19 which supply at least propane to the reactor.

In the provision unit 110, the dehydrogenation reaction typically takes place at low pressure such as, for example, 50 kPa to −500 kPa. The feed flow is compressed upstream of the heat exchanger 120 with a compressor, which can be part of the provision system 110, to a final pressure of 1 MPa to 1.8 MPa. There may also be some fine purification steps of the gas, for example the removal of H2S, water and chlorine.

Irrespective of the origin thereof, the feed flow 1 comprising at least hydrogen and C3 is fed to the hot heat exchanger 120 and cooled therein against other material flows. For example, the feed flow 1 is fed to the hot heat exchanger 120 at a high temperature level which substantially corresponds to a natural ambient temperature of, for example, between 10° C. and 40° C., in particular between 15° C. and 25° C. and is withdrawn therefrom at an average temperature level in a range from −10° C. to −40° C., for example at an average temperature level of −15° C., −25° C. or −35° C., as a cooled feed flow 2. As a result of the cooling in the heat exchanger 120, part of the components of the feed flow 1 condenses so that the cooled feed flow is a mixture of at least one liquid and a gas. The cooled feed 2 is fed to a gas separator 142 and is separated there into a first residual gas flow 3 and a first condensate flow 7. Due to the different vapor pressures of hydrogen and C3, the first residual gas flow 3 is enriched in hydrogen and depleted in C3 with respect to the feed flow 1, while the first condensate flow 7 behaves exactly vice versa. The first residual gas flow 3 is fed to the cold heat exchanger 130 and is cooled there from the average temperature level to a low temperature level (also referred to as low temperature level within the scope of the disclosure), which is, for example, in a range from −80° C. to −110° C. In this case, a part of the components of the first residual gas flow 3 is again condensed and a supercooled residual gas flow 4 is thus withdrawn from the cold heat exchanger 130, which contains at least one liquid phase and a gas phase.

The supercooled residual gas flow 4 is separated in a second gas separator 144 into a second residual gas flow 5 and a second condensate flow 8. The second residual gas flow 5 is enriched with hydrogen and depleted in C3 in relation to the first residual gas flow 3, while the second condensate flow 8 is depleted in hydrogen and enriched in C3 with respect to the first residual gas flow 3.

In the example shown, the second residual gas flow 5 is supplied to an expander or a turbine, which in this case is designed for example as a turbine 150. An expanded residual gas flow 6 is withdrawn from the turbine 150, which flow is in turn partially liquefied due to the energy released in the turbine 150. In a third gas separator 146, the expanded residual gas flow 6 is again separated into a third residual gas flow 11 and a third condensate flow 9. The third condensate flow 9 is again depleted in hydrogen and enriched in C3 with respect to the second residual gas flow 5, the third residual gas flow 11 is accordingly enriched in hydrogen and depleted in C3 with respect to the second residual gas flow 5. According to the invention, however, the use of the expander is optional. Alternatively, the turbine 150, the gas separator 146 and the condensate flow 9 are omitted. Accordingly, the residual gas flows 5 and 11 would be identical. As a further alternative, the expander 150 could simply be designed as a throttle valve.

The three different condensate flows 7, 8 and 9 are combined, with heating, if appropriate, in the cold heat exchanger 130, at the average temperature level, into a fourth gas separator 148, in which a fourth residual gas flow, which evaporates at the set conditions, which is here referred to as flash gas flow 12, is separated. A liquid product flow 10, which consists essentially of C3, is withdrawn on the side of the liquid phase and conveyed via a pump 149 at least through the hot heat exchanger 120.

A part of the third residual gas flow 11, which consists primarily of hydrogen, is heated by the cold and hot heat exchanger 130, 120 and withdrawn as a gas product 20. In some embodiments, part of the gas product 20 can be returned to the provision unit, in particular for purging purposes, for example during a catalyst regeneration. A further part 14 of the third residual gas flow 11 is backmixed with a part 13 of the second condensate flow 8 under expansion of the two flows, heated/evaporated as internal refrigerant 15 via the cold heat exchanger 130 and backmixed upstream of the hot heat exchanger 120 with the flash gas 12 and further heated as mixed flow 17 in the hot heat exchanger 120. The mixed flow 17 is preferably recycled into the feed flow 1 in order to increase the overall yield of C3 in the liquid product flow 10.

As already mentioned, the reactor 110 requires a raw material flow 19 which contains propane. In the example shown, this raw material flow is initially provided as a liquid raw material flow 18. The liquid raw material flow 18 is cooled in the hot heat exchanger 120 and optionally also in the cold heat exchanger 130 and mixed with a third part 16 of the third residual gas flow 11 before it is heated again as a mixed raw material flow 19 via the hot heat exchanger 120 or both heat exchangers 130 and 120 and fed into the reactor 110. In particular, the liquid raw material flow 18 is expanded after the supercooling in the hot heat exchanger 120 and/or the cold heat exchanger 130, for example via one or more throttle valves. As a result of the expansion of the supercooled liquid raw material flow 18 and the addition of residual gas flow (16) at low temperature, thermal energy from the liquid raw material flow 18 is converted into volume work so that the mixed raw material flow 19 is at a temperature level that is substantially lower than that of the corresponding supercooled liquid raw material flow 18, before the heating in the respective heat exchanger 120, 130. The evaporation heat of the liquid raw material flow 18 is thus used as the main cold source for the partial condensation of the feed flow 1 and the first residual gas flow 3. The additional useful cooling of the internal refrigerant 15 is decisive for the cold heat exchanger 130, but evaporation heat can also be withdrawn here by the supercooled liquid raw material flow 18.

The liquid raw material flow can be provided, for example, at a pressure level in a range from 1.5 MPa to 2.5 MPa. Downstream of the heat exchanger(s), the gaseous raw material flow 19 is, for example, at a pressure level in the range from 200 kPa to 500 kPa.

Downstream of the reactor and upstream of the compressor of the provision unit 110, the feed flow 1 is present, for example, at a pressure level in the range of about 100 kPa to 200 kPa, downstream of the compressor and upstream of the hot heat exchanger 120 at a pressure level in the range of, for example, 1 MPa to 1.8 MPa.

In principle, all flows have a correspondingly higher pressure upstream of valves due to pressure losses through the respective valve than the respective flows downstream of the respective valve. In particular, an explicitly described pressure drop or a pressure drop resulting from the different pressure levels of flows transferred into one another is realized by the respective valve.

Overall, at least the cold heat exchanger 130 operates without an externally cooled refrigerant. The cooling power required here is thus provided via the pressure difference between feed flow 1 (or the residual gas flow 3 remaining from it) on one side and the mixed raw material flow 19 and the internal refrigerant 15 on the other side. To increase the pressure of feed flow 1, one or more compressors can be provided upstream of the hot heat exchanger 120 ( ).

If necessary, an external refrigerant 21 can be used at the average temperature level. In this way, variable amounts and/or temperatures of the feed flow 1 or of the liquid raw material flow 18 can be compensated.

As already mentioned, the system 100 can also be used to separate a feed flow which comprises at least hydrogen and a hydrocarbon (C4) comprising four carbon atoms per molecule, in particular 1-butene, 2-butene, 1,3-butadiene and/or butane. The features and advantages of system 100 described above, which apply to the separation of a feed flow 1 comprising C3, correspondingly apply to C4. However, the set temperature levels can differ.

It should be mentioned that multiple exchangers can also be used in each cooling step, depending on the energy balance and demand, i.e., the respective flows to be cooled and heated can also be distributed to 2, 3, 4 or more exchangers.

Furthermore, it should be noted that the method is not limited to the two cooling steps explained here, i.e., one, two or more intermediate temperature levels (such as at about −50° C. to −90° C.) and one, two or more additional heat exchangers and corresponding separation device can be additionally used.

1. A method for separating a feed flow which contains at least hydrogen and a hydrocarbon with three to four carbon atoms per molecule, which feed flow is formed from a raw material flow,
wherein the feed flow is partially liquefied in the compressed state using at least two coolers which are operated at different temperature levels, via at least two cooling steps, to obtain at least two condensate flows and at least two residual gas flows,
wherein the residual gas flow of a cooling step is respectively fed into the subsequent cooling step, and wherein each condensate flow is depleted in hydrogen and enriched in the hydrocarbon with respect to the feed flow and each residual gas flow is enriched in hydrogen and depleted in hydrocarbon with respect to the feed flow,
wherein
a part of at least one of the condensate flows is combined with a part of at least one of the residual gas flows under expansion and used as internal refrigerant for at least one of the cooling steps or coolers and is at least partially recycled to the feed flow.

2. The method according to claim 1, wherein at least a first cooling step takes place at an average temperature level in a range from −40° C. to +10° C., preferably from −40° C. to −10° C., and at least a second cooling step takes place at a low temperature level in a range from −130° C. to −80° C., preferably from −110° C. to −90° C.

3. The method according to claim 2, wherein only material flows formed from the raw material flow are used to achieve at least the low temperature level.

4. The method according to claim 2, wherein a refrigerant which is not provided internally in the process is used to achieve the average temperature level.

5. The method according to claim 2, wherein the first and second cooling steps are performed by counterflow heat exchange.

6. The method according to claim 2, wherein the residual gas flow of the second cooling step is subjected to at least one expansion to obtain further condensate flows and residual gas flows.

7. The method according to claim 1, wherein the internal refrigerant is formed of a part of at least one of the condensate flows and a part of at least one of the residual gas flows, wherein the condensate flow is formed in a step which takes place upstream of the step in which the residual gas flow is formed.

8. The method according to claim 1, wherein a plurality of or all condensate flows are each at least partially combined to form a combined flow, and the combined flow is subjected to a gas separation to form a flash gas and a liquid product flow.

9. The method according to claim 1, wherein the liquid product flow, a predominantly hydrogen-containing gas product flow, formed from a part of at least one of the residual gas flows, and the flash gas are heated to a temperature level corresponding to the temperature level of the feed flow.

10. The method according to claim 1, wherein the flash gas is at least partially recycled into the feed flow.

11. A system for separating a feed flow which contains at least hydrogen and a hydrocarbon with three to four carbon atoms per molecule,
having at least two heat exchangers, of which at least one can be operated at an average temperature level and at least one can be operated at a low temperature level, and which are configured to cool the feed flow according to the counterflow principle, and
having at least two phase separation devices, which are each configured to split a partially liquefied flow into a condensate flow and a residual gas flow,
wherein
means configured to combine a part of at least one of the condensate flows with a part of at least one of the residual gas flows under expansion, and to feed the internal refrigerant to at least one of the heat exchangers.

12. The system according to claim 11, also having means which enable the system to carry out a method for separating a feed flow which contains at least hydrogen and a hydrocarbon with three to four carbon atoms per molecule, which feed flow is formed from a raw material flow,
wherein the feed flow is partially liquefied in the compressed state using at least two coolers which are operated at different temperature levels, via at least two cooling steps, to obtain at least two condensate flows and at least two residual gas flows,
wherein the residual gas flow of a cooling step is respectively fed into the subsequent cooling step, and wherein each condensate flow is depleted in hydrogen and enriched in the hydrocarbon with respect to the feed flow and each residual gas flow is enriched in hydrogen and depleted in hydrocarbon with respect to the feed flow,
wherein
a part of at least one of the condensate flows is combined with a part of at least one of the residual gas flows under expansion and used as internal refrigerant for at least one of the cooling steps or coolers and is at least partially recycled to the feed flow.
